(19) United States
(12) Patent Application Publication
(10) Pub. No.: US 2005/0143357 A1
(43) Pub. Date: Jun. 30, 2005

(54) Vitamin d upregulated protein 1 (vdup-) methods and uses thereof

(76) Inventors: Ake Pousette, Alvsjo (SE); Gunnar Norstedt, Forfattarvagen (SE); Jacob See-Tong Pang, Taipei (TW)

Correspondence Address: BROWDY AND NEIMARK, P.L.L.C.;624 NINTH STREET, NW, SUITE 300, WASHINGTON, DC, 20001-5303, US

(21) Appl. No.: 10/505,665

(22) PCT Filed: Feb. 25, 2003
(86) PCT No.: PCT/SE03/00301

(30) Foreign Application Priority Data
Feb. 25, 2002 (US) 60358706

(51) Int. Cl. C12Q 1/68; C07H 21/04; A61K 31/59; C07K 14/72

(52) U.S. Cl. 514/167; 435/6; 435/69.1; 435/320.1; 435/325; 530/350; 536/23.5

(57) ABSTRACT

A method for selecting a compound or a composition of matter for treatment of hyperproliferative disorders of the prostate in a mammal by administering the compound or the composition to a mammal (in vivo) or to a prostate cell culture (in vitro) and assessing the effect of the compound or the composition on the content of the transcription and/or translation product of a gene under the influence of at least one regulatory region of Vitamin D upregulated protein 1 (VDUP-1) gene and optionally assessing the effect of the compound or the composition on the content of apoptosis signalling kinase (ASK-1) and/or thioredoxin, in the prostate cells of said mammal or in the cells of the prostate cell culture. A substance for use in the treatment of hyperproliferative disorders of the prostate selected by the above method, a pharmaceutical composition comprising said substance, a method of treatment by of a hyperproliferative disorder of the prostate. A gene encoding VDUP-1 or a functional equivalent thereof for use as a medicament and a vector capable of expressing VDUP-1. A method of diagnosis of a hyperproliferative disorder of the prostate in a mammal by assessing the content of the transcription and/or translation products of at least one of the genes encoding ezrin, thioredoxin, peroxiredoxin 5, cyclin-dependent kinase 4, growth response protein CL6, defender against cell death 1, CD24, osteoactivin, vitamin D-upregulated protein 1, placental growth factor gene 1+2, Gata-4, duffy blood antigen, interferon-inducible 17-kDa membrane protein, 14-3-3 protein, CD9 antigen (p24), Ste-20 related kinase, ephrin type A receptor 5 precursor, programmed cell death factor 4 and DNA-methyltransferase 3a, in cells obtained from the prostate of said mammal. A kit for use in a method of diagnosis of a hyperproliferative disorder of the prostate in a mammal.

### FIELD OF THE INVENTION

This invention relates to disease diagnostics and the use of pharmaceutical drug targets to influence prostate disorders. Specifically, the invention relates to thioredoxin and vitamin D-up regulated protein 1 as essential components in causing effects via androgens/steroids on the prostate.

### BACKGROUND OF THE INVENTION

Disorders of the prostate in the form of hyperplasia or cancer are commonly occurring in men. A widely used biochemical marker for prostate dysfunction is PSA (prostatic specific antigen). Serum values of PSA are often increased in prostate cancer. Frequently one analyses the content of androgen receptors in prostate cancer, which can indicate modalities for drug treatment. Surgical intervention offers one way to treat prostate cancer but normally therapy also includes pharmaceuticals and such agents often interfere with steroid synthesis or actions. Over all there is a need for improvements both in the area of diagnostic markers to better classify disorders of the prostate or to predict prognosis or to select and follow therapy. There is also a need for improvements of therapy of prostate disorders as many presently used procedures suffer from difficult side effects or poor efficacy.

Prostate gland depends on androgens for its growth, differentiation and function. Underdeveloped prostate gland is seen in eunuchs who lack androgen stimulation since childhood. In experimental animals, castration induced androgen-ablation regress the prostate gland via an active process of apoptosis of the epithelial cells. Apoptosis can be observed within one day of castration and nearly ⅔ of epithelial cells are lost in the ventral prostate by seven days of castration. In contrast, testosterone-replacement to castrated rats stimulates the re-growth of the gland to its normal size via proliferation of new epithelial cells from basal cells. A balance between these two contrasting cellular processes is important for maintaining the homeostasis of prostate gland. Yet, the underlying mechanisms for this regulation are still far from elucidated.

Testosterone is the main circulating androgen secreted primarily by Leydig cells in testis but is also secreted from the adrenal gland and formed by peripheral conversion of adrenal steroid. Testosterone is mainly bound to albumin and sex-hormone-binding globulin and only a small fraction is dissolved freely in the serum. Once it enters prostate cells, about 90% is converted to dihydrotestosterone (DHT) by the enzyme 5 α-reduotase. DHT, which has fivefold higher binding affinity for the androgen receptor (AR) than testosterone, can dissociate AR from heat-shock protein and phosphorylate the receptor. Subsequently, AR dimerizes and binds to androgen-response elements in the promoter regions of target genes. Co-activators and co-repressors may interact with the AR complex and the general transcription apparatus to stimulate or inhabit target gene transcription.

### SUMMARY OF THE INVENTION

According to a first aspect the present invention aims at providing a method for selecting a compound or a composition of matter for treatment of hyperproliferative disorders of the prostate in a mammal. This object is achieved by administering the compound or the composition to a mammal (in vivo) or to a prostate cell culture (in vitro) and assessing the effect of the compound or the composition on the content of the transcription and/or translation product of a gene under the influence of at least one regulatory region of Vitamin D upregulated protein 1 (VDUP-1) gene and optionally assessing the effect of the compound or the composition on the content of apoptosis signalling kinase (ASK-1) and/or thioredoxin, in the prostate cells of said mammal or in the cells of the prostate cell culture.

Further aspects of the invention and embodiments thereof are as defined in the claims.

### DETAILED DESCRIPTION OF THE INVENTION

Based on a substantial investigation we have arrived at a mechanism whereby steroid hormones influence key functions of the prostate and we have realized that this knowledge, unexpectedly, can be used to test, develop and/or search for new modalities of treatment of hyperproliferative disorders of the prostate such as prostate hyperplasia and prostate cancer. The pathway we have identified can be summarized as follows:
Sex-steroids/prolactin→↓Vitamin D up-regulated protein 1→↑Thioredoxin→Inactivation of ASK-1→↑cell growth
Anti sex-steroids/Vitamin D→↑Vitamin D up-regulated protein 1→↓Thioredoxin→Activation of ASK-1→↓cell growth

The above scheme is to be understood as:

In the prostate cells, stimulation by sex-steroids and/or prolactin is coupled with a decrease in the content of vitamin D unregulated protein 1, which is coupled with an increase in the content of thioredoxin, which will result in enhanced cell growth, part of which depends on the inactivation of ASK-1. Stimulation by anti sex-steroids and/or vitamin D will have the opposite effect. The above scheme will be referred to as the pathway.

The various components of the above pathway are all known from the prior art. Accession numbers are e.g. for VDUP-1: NP_{—}006463. [gi:7949163]; for thioredoxin: BAA05742.[gi:4433244], and for ASK-1: Q99683 [gi:6685617]. The database entries above concern the human components. The present invention also includes the corresponding components in other species as well as related homologues and/or natural or experimental variants of the components. These are described as functional equivalents.

Steroid hormones such as androgens, estrogens, glucocorticoids bind receptors on the prostate as do vitamins like vitamin D and the so-called retinoic acids. A large variety of such compounds, herein defined ligands of the steroid receptor family (LSR), include both agonists and antagonists of steroids acting on steroid receptors or on fat-soluble vitamin receptors acting on steroid related receptors or presently unknown compounds that react with so called orphan steroid receptors. Such compounds can, in some cases, influence prostate cell growth either to enhance growth or to retard growth.

It is previously well known that androgens stimulate growth of the prostate and that anti-androgens reduce growth. In clinical practice this has lead to treatments of prostate cancer either involving receptor antagonist or agents it reduce the level of bioactive androgens. Not only androgenic substances influence prostate growth but also estrogens, progestins and protein hormones such as prolactin and growth hormone.

A variety of agents reduce prostate growth, at least using model cell systems and this can be exemplified by the previous finding that vitamin D induce apoptosis of prostate cells. Vitamin D up-regulated protein 1 (UP-1, also known as thioredoxin interacting protein TXNIP) is increased by vitamin D as previously demonstrated in non-prostatic cells. One function of VDUP-1 is to block thioredoxin. Thioredoxin is ubiquitously expressed in cells and has many functions related to redox state and the action of this enzyme has consequences for basic cellular functions such as cell growth. Thioredoxin is also known as a direct cell-death inhibitor through the blocking of a cell-death signalling pathway involving ASK-1 (apoptosis signalling kinase or MEK kinase 5).

The present invention provides new information that puts the building blocks described above in a coherent pathway of relevance for prostatic cells and this can be used for pharmaceutical development. Our findings are that VDUP-1 protein functions as a “switch protein” in the prostate; a high level of VDUP-1 and low levels of thioredoxin is a feature of suppressed cell growth and a low level of VDUP-1 and high levels of thioredoxin is a marker of increased growth. The present invention uses this information on the dichotomy of VDUP-1, thioredoxin and ASK-1 regulation to develop better pharmaceuticals to influence prostate growth.

Persons skilled in the art know of procedures to develop low molecular weight compounds that either enhance or inhibit actions of steroid/vitamin receptors or the related orphan receptors. The compounds to be tested can be of various nature. For example, in one application cells are exposed to low molecular weight drug-like compounds that are analogues to steroids or diverse forms of other lipid-like molecules. The choice of molecules for screening can be carried out using so called chemical libraries based on different types of compounds. Procedures to synthesize organic molecules and to generate libraries of diverse chemical compounds are known to persons skilled in the art.

An important feature of this aspect of the present invention is that not only can it be used to define and optimize one type of receptor interacting compound but also to identify combinations of different types of compounds.

Put in the context of the present invention, measurements of VDUP-1 protein or mRNA can be used to search for compounds that are optimized in activity of increasing VDUP-1. For certain applications, measurements of downstream components i.e. thioredoxin and ASK-1 serve a purpose. Such procedures can, for example, use a prostate cell system where compounds are tested for their ability to increase VDUP-1.

There are many different techniques to measure VDUP-1 or other components of the pathway and such techniques can be exemplified by immuno-assays, hybridization based techniques to measure protein or mRNA.

A prostate cell system can be any type of cell derived from the prostate of human and animal origin and be in the form of primary cells or in the form of established cell lines. Such systems and such cell lines are well known to persons skilled in the art and one source to obtain cell lines is the American Tissue Culture Collection (ATCC).

For certain applications the above mentioned pathway will be monitored in the in vivo situation where prostate tissue will be obtained (biopsies) and used for analysis.

A cell system for use according to the invention may be further simplified by using so called reporter genes where the regulatory portion of the VDUP-1 gene regulates a reporter that is easy to measure. The regulatory portion of the VDUP-1 consists of an up-stream regulatory DNA sequence that can contain up to 10-100 kb DNA. Reporter genes can have different designs. Normally they are coding regions, corresponding to a gene product, linked to the regulatory DNA sequence of interest. The gene product of a reporter gene is normally easy to measure and can e.g. be luceferase, beta galactosidase, green fluorescent protein or the enzyme CAT. Reporter genes are transferred into cell by different means e.g. using liposomes, membrane disrupting agents, viral vectors or injections and can be used either transiently or as stable integrants in the cellular genome. The purpose of rung reporter genes is to facilitate measurements of agents that influence gene expression.

In one embodiment a reporter gene assay can be used to define and titrate androgen antagonists, vitamin D agonists and Prl receptor antagonists. The Prl receptor antagonists of relevance include not only compounds acting on the receptor itself but also compounds that act downstream of the receptor to influence e.g. the JAK-STAT-SOCS system (Janus kinase-signal transducer and activator of transcription-suppressor of cytokine signaling). Androgen antagonists are known in the form of e.g. GnRH analogues leuprolide, goserelin and histrelin; GnRH antagonist teverelix, abarelix and cetrorelix; antiandrogen flutamide and cyproterone acetate; and 5 alpha reductase inhibitor finasteride. Vitamin D agonists can be ergocalciferol, calcifediol, calcitriol, dihydrotachysterol. Prl receptor antagonist can be in the form of protein mutants that fail to dimerize receptors. JAK-STAT-SOCS can be blocked by tyrosin kinase inhibitors or agents that eliminate expression, such as antisense compounds.

Based on the findings of the drug screening method according to the invention, the present invention provides a pharmaceutical composition for use in the treatment of hyperproliferative disorders of the prostate, comprising at least one compound or composition of matter selected in the screening method. The person skilled in the art of pharmaceutical sciences will know how to formulate a drug for use in the required therapy, for the treatment of a mammal suffering from a hyperproliferative disorder, such as prostate cancer or hyperplasia, using conventional formulation aids in view of the route of administration. Also, optionally the pharmaceutical composition may comprise may other suitable active ingredients, provided these are compatible with the former ones.

In a method of therapy according to the invention, a medicament according to the invention is administrated to a mammal in need of such therapy at a regimen to be determined by the attending physician.

The invention can be used both to discover new chemical entities that influence the pathway and to make compositions of existing compounds. A particularly useful composition for the treatment of prostatic disorders is an androgen antagonist combined with vitamin D. To find out how much of each component one should use one can first add anti-androgens in different concentrations to cells and then measure components of the pathway e-g. VDUP-1 at different time intervals. Using an anti-androgen concentration that only cause a partial effect on VDUP-1 one can add different concentrations of vitamin D (or similarly acting compounds) and use the same readout as before. The concentration of vitamin D that causes the same effect of a high concentration of anti-androgens can thereby be determined. This type of composition can further be tested using different types of cell systems and should also be combined with readouts like ASK-1 and or cell growth/apoptosis. Obviously the goal is to arrive at a composition containing both components that show maximal efficacy. The utility of this would be to translate the experimentally found ratio between anti-androgen and vitamin D and convert his into a pharmaceutically acceptable composition for treatment in humans. Such a calculation can be aided by previous information on serum levels after the intake of anti-androgens or vitamin D. Although this exemplifies the use of anti-androgen and vitamin D is will be obvious to persons skilled in the art that a similar approach to optimize combinations is applicable for other types of compounds active on the pathway.

Another embodiment of the present invention is to increase the content on VDUP-1 in a prostate cell to reduce its growth potential. This can be achieved by so called gene therapy where the gene encoding VDUP-1 is delivered to prostate cells. Techniques to carry this out include viral infections and are known by persons skilled in the art. The essential part is the use of a vector that will deliver VDUP-1, or other components of the pathway, into prostate cells in such a manner that the protein will be expressed. Vectors for gene transfer include, in an unrestricted manner, adenovirus or shorter forms of DNA that can be used to infect or transfect the prostate in situ using systemic of local modes for gene transfer.

An increased cellular content of VDUP-1 may also be achieved by other means. Thus, in one embodiment of the present invention an increased cellular content is achieved by extending the half life of VDUP-1. In this case, the VDUP-1 protein suitably is monitored by immunological techniques in time course studies. Compounds that will extend the half-life of the protein can be found in a screening procedure, as principally known to the person skilled in the art. Such compounds may, for example, be interfering with the ubiquitination pathway. Compounds that exert effects on this pathway include inhibitors of ubiquitin ligases and proteasomal inhibitors.

Another embodiment of the present invention is to enhance the inhibitory action of VDUP-1 on thioredoxin. The screening strategy can here be either a cell based screen where the readout is thioredoxin content or alternatively be a screen in vitro based on protein-protein contacts of recombinantly produced VDUP-1 and thioredoxin. The latter, optionally combined with a structure based design strategy, can be used to assay new compounds with the ability to increase the inhibitory action of VDUP-1 on thioredoxin.

The pathway described in the present invention is not only related to steroid action but also to other parameters that influence the pathway. Our finding that reduction of androgens generates a situation of cellular stress, in particular oxidative stress, which act on the pathway leading to ASK-1 is of high relevance to explain the situation that prostate growth is increased in aged men although serum levels of androgens are low. The utility of this is that androgen treatment can be of preventive value if androgens are given at a dose sufficient only to reverse the oxidative stress. The present invention gives the opportunity to test and titrate such a concept in the purpose to prevent prostatic disease.

According to one aspect, the present invention provides a therapeutic method for preventing and/or curing hyperproliferative disorders of the prostate by reducing oxidative stress, induced by androgen deficiency, using agents that specifically influence pathways involved in oxidative stress. Such agents can be exemplified by a large variety of synthetic or naturally occurring anti-oxidants. The present invention offers a test system to investigate such compounds for interference in androgen dependent pathways and this information may later be used in a preventive or therapeutic purpose. An embodiment of the present invention is therefore to monitor the VDUP-1, thioredoxin, ASK-1 pathway in cells for stress effects, as measured by analysis of stress markers, caused by androgen withdrawal and to screen for substances that reduce the stress effect.

A particularly useful combination of treatment is to use low levels of androgens in combination with anti-oxidants including, but not restricted to, vitamin C, vitamin E, beta carotene, grape seed extract, vitamin A, selenium, and coenzyme Q10. The present invention offers a system to define the optimal composition e.g. the combination of low androgens and vitamin E. In this embodiment of the invention, androgen will be added to cells as described above to determine a dose that block VDUP-1 and increase growth. The androgen will then be removed or its concentration reduced and different concentration of vitamin E will be added (or will be present throughout the experiment). The effects of these compounds will be monitored in controlled experiments by the analysis of the pathway as described above. The method offers a model to determine and screen for effects of anti-oxidants in situations of androgen deficiency. The situation that anti-oxidants like vitamin E modulate stress induced by androgen deficiency can be use in a therapeutic context. The doses and compositions can be worked out using the present invention and this can be translated into a composition for therapeutic use using procedures described above. The route of administration of such composition will in the preferred case be oral but other routes are possibly such as a local deposition in prostate tissue. The preferred dose regime is daily but may vary dependent on kinetics or on individual basis. The manufacture of pharmaceutical steroids and vies are known to experts in the field as are procedures to create chemical libraries.

In any type of drug development there is a need to follow effects using several markers that reflect the cellular process one seeks to influence. Such markers can also have an additional use as a diagnostic tool to be used on patient material. Persons skilled in the art know that a variety of different assay techniques can be used for this purpose. These can be exemplified by techniques to analyse DNA (gene deletions and/or gene variability) and/or mRNA (levels) encoding the marker or techniques to analyse the protein component using e.g. immunological techniques. According to one aspect of the invention, several new markers for this purpose are provided, to be used either in isolation or in a combination, to define androgen response or androgen dependency in the prostate in the purpose to facilitate diagnosis or drug development processes.

Suitable markers for use according to the invention are: Ezrin, thioredoxin, peroxiredoxin 5, cyclin-dependent kinase 4, growth response protein CL6, defender against cell death 1, CD24, osteoactivin, vitamin D-upregulated protein 1, placental growth factor gene 1+2, Gata-4, duffy blood antigen, interferon-inducible 17-kDa membrane protein, 14-3-3 protein, CD9 antigen (p24), Ste-20 related kinase, and ephrin type A receptor 5 precursor, programmed cell death factor 4 and DNA-methyltransferase 3a.

The markers can be analysed using any or the techniques described above using in vitro systems consisting of prostatic cells, animal models or patient samples to evaluate effects of drug-like molecules or to refine diagnosis of prostate disorders.

According to one aspect of the invention, agents to assess the presence and level of said markers may be used in the form of a kit to facilitate the diagnosis of prostate disorders. Such agents may be e.g. antibodies directed against at least one of the markers.

More generally, a kit for use in a method according to the invention may comprise any means suitable for determining e.g. the level of at least one transcription and/or translation product of one or more of the genes of interest according to the invention, as herein defined.

The invention will be better understood by referring to the following illustrative, non-limiting examples.

### EXAMPLES

1. The level of VDRP1 is decreased by androgen and increased by androgen withdrawal Micro array experiments were conducted using a rat prostate model. In this experiment a group of rats were castrated after which prostate RNA was isolated using the Trizol method. Another group of castrated rats received androgen treatment. The RNA was used to measure VDIP-1 mRNA. Information on how to carry out micro array hybridization and how to carry out the rat experiment can be found in the publication by Pang ST et al (Endocrinology, 143(12):4897-4906). The results show that administration of androgen reduces VDUP-1 mRNA and androgen removal has the opposite effect to increase VDUP-1 mRNA and this covaries with growth and growth reduction of the prostate. The micro array experiments also led to two other findings namely, the realization that androgen withdrawal creates cellular stress and that markers for androgen dependency can be found. Below is a record of the interpretation of gene expression profiles.

A Gene Expression Profile that Indicate a Stress Response Following Androgen Removal.

Gene expression was analyzed in rat ventral prostates of 1, 4 and 8 days-castrated rats (C1, C4 & C8), and of 1 and 4 days testosterone-replacement rats (T1 & T4). For the purpose of comparing the gene expression in two experimental conditions, 238 genes or about 8% of the total transcripts that passed the filter criteria (i.e. 2-fold differences in expression and no missing data in any time point) were selected for fierier analysis. These genes were analyzed in parallel using clustering analysis and six different clusters of genes that followed a similar pattern of expression could be distinguished. It was clear that reduction of androgens i.e. castration changed expression of genes that play a role in oxidative stress. Examples of such genes and comments regarding functional roles are as follows: thioredoxin, peroxiredoxin 5 and 1 (PRX 1 and 5), and glutathione peroxidase 1 (GPX1) are genes that functionally related to cellular redox regulation and were down-regulated after castration. Alteration of antioxidant proteins can cause oxidative stress to cells since the balance of reactive oxygen species and antioxidant system is lost. The finding indicates that oxidative stress may be one of the initiation factors for androgen-ablation induced prostate apoptosis and this is further supported by the finding that GST (glutathion-S-transferase) was up-regulated during androgen-ablation induced prostate apoptosis. The androgen dependency of several antioxidant genes is here described for the first time. Since oxidative stress and loss of GST expression is a risk factor for prostate cancer development, the present observation provides clues to understanding prostate tumorigenesis. Perhaps this could also explain the paradoxical epidemiological finding of prostate cancer occurring during the period of life when testicular function is declining and levels of testosterone are falling.

Identification of Androgen-Regulated Genes

Gene expression that fluctuated with the circulating testosterone level was regarded as expression of androgen-regulated genes. In total, 48 androgen-induced genes and 10 androgen-repressed genes were identified. This experiment was complemented by gene expression profiles from androgen responsive and non-responsive LNCaP cells. Put together we have selected certain genes that will fulfil a marker function of steroid responsivity. These were: Ezrin, thioredoxin, peroxiredoxin 5, cyclin-dependent kinase 4, growth response protein CL6, defender against cell death 1, CD24, osteoactivin, vitamin D-upregulated protein 1, placental growth factor gene 1+2, Gata-4, duffy blood antigen, interferon-inducible 17-kDa membrane protein, 14-3-3 protein, CD9 antigen (p24), Ste-20 related kinase, and ephrin type A receptor 5 precursor, programmed cell death factor 4 and DNA-methyltransferase 3a.

Apoptosis and Androgen Dependent Genes

Another intriguing observation based on the above described experimental procedure is the identification of distinct signaling pathways that may be involved in androgen-ablation induced prostate apoptosis. In addition to its function in cellular redox status, TRX is also known as an antiapoptotic factor. TRX can directly block the apoptosis-signalling pathway delivered through a mitogen-activated protein kinase kinase kinase (MAPKKK) family member—apoptosis signal-regulating kinase 1 (ASK 1). ASK 1 can be activated by oxidative stress after dissociated from its inhibitor TRX. The present inventors found that TRX is down-regulated after androgen-ablation induced prostate apoptosis. In addition, rat gene clone N27, which is homologous to human vitamin D up-regulated protein 1 (VDUP 1) gene and a negative regulator of TRX, was highly induced after castration. Taken together, this indicates that TRX function might have been suppressed by repressing its gene expression and increasing the expression of its antagonist—VDUP1. Apparently, this finding supports the involvement of ASK 1 signalling pathway in androgen-ablation induced prostate apoptosis. Furthermore, we also find supportive evidences to this assumption. In addition, the present inventors found that the expression of ezrin, a membrane-cytoskeleton linker that can signal cell survival through the phosphatidylinositol 3-kinase/Akt pathway (P13K/Akt), was down-regulated after castration. As Akt can decrease ASK 1 kinase activity that is stimulated by oxidative stress, it would be rational to speculate that suppressing the P13K/Akt surviving pathway by down-regulating the expression of ezrin can facilitate the apoptosis process triggered by androgen-ablation.

2. Increased Levels of VDUP-1 Cause Apoptosis in Human Cells

Human prostate cancer cell lines LNCaP, DU146 and PC3 were transfected with a DNA construct containing VDUP-1 cDNA under control of CMV promoter using lipofection as a transfection agent. As a control we used a promoter construct that did not express VDUP 1. After 3 hours of transfection and 14 hours of observation after transfection, the degree of apoptosis was measured using Annexin and propidium iodide assessment and cell counting by trypan blue exclusion. The results show that overexpression of VDUP-1 significantly caused an increased level of apoptosis in these prostate cancer cell lines.

3. A Cell Based Screening Assay to Analyse Agents that Influence VDUP-1 Regulation

A human cell line, LNCaP was transiently transfected with a DNA vector composed of the upstream regulatory portion of the VDUP-1 fused to the gene encoding luceferase. The procedures to carry out transfection were essentially as described in example 2. At different time points a steroid with androgen properties, exemplified as R1881, were added to cell cultures, as was calcitriol (vitamin D). At different time points, luciferace activity in cell extracts was measured using a luminometer. The outcome of this experiment was that the level of luceferase was decreased by the steroid and was increased by vitamin D.

4. A Principal Demonstration that the JAK-STAT-SOCS Pathway Influence VDUP-1

The human cell line LnCAP was transfected with expression vectors for different types of SOCS. Following transfection the readout was immunodetection of VDUP-1 protein using Western blots. It was found that SOCS, to varying degree depending on dose and type of SOCS, could decrease the level of VDUP-1. This demonstrates that agents acting on different pathways than steroids influence VDUP-1.

5. A Principal Demonstration that ASK-1 is Involved in a Pathway that Links Androgens and Stress to Apoptosis.

Ventral prostates were obtained from normal, 1 and 3-days castrated rats. Cellular extracts were, made and ASK-1 activity was measured by immunoprecipitation for Western blots and Kinases assay. The results showed that ASK-1 was activated after castration. The term activation refers to the ability to distinctly measure ASK-1 phosphorylation. The level of oxidative stress was also determined by carbonyl content in oxidatively modified protein using the 2,4-Dinitrophenylhydrazine method. It was shown that a higher level of oxidatively modified protein was present in castrated prostate.

6. Diagnostic Use of Markers Defined in the Present Application

The markers defined in the present application were analysed using measurements of RNA levels using a quantitative PCR method (light cycling, DNA chips, DNA mutational analysis or immuno-histochemistry. The samples that were tested were either extracts from prostate cells or tissue sections of human prostate or cells from prostate in to form or LNCaP or LNCaP-r (subline of LNCaP and is resistant to androgen). The panel of tested substances included: Ezrin, thioredoxin, peroxiredoxin 5, cyclin-dependent kinase 4, growth response protein CL6, defender against cell death 1, CD24′ osteoactivin, vitamin D-upregulated protein 1, placental growth factor gene 1+2, Gata-4, duffy blood antigen, interferon-inducible 17-kDa membrane protein, 143-3 protein, CD9 antigen (p24), Ste-20 related kinase, and ephrin type A receptor 5 precursor, programmed cell dead factor 4 and DNA-methyltransferase 3a. These were regarded as markers based the following criteria: androgen regulated at the mRNA level, mutated in androgen resistant cells, immuno-detected in a disease related manner. The use of these markers can be to analyse the markers at the DNA, RNA or protein level to diagnose a prostate disorder, and to predict prognosis and progression of prostate cancer.

1. A method for selecting a compound or a composition of matter for treatment of hyperproliferative disorders of the prostate in a mammal by administering the compound or the composition to a mammal (in vivo) or to a prostate cell culture (in vitro) and assessing the effect of the compound or the composition on the content of the transcription and/or translation product of a gene under the influence of at least one regulatory region of Vitamin D upregulated protein 1 (VDUP-1) gene and optionally assessing the effect of the compound or the composition on the content of apoptosis signaling kinase (ASK-1) and/or thioredoxin, in the prostate cells of said mammal or in the cells of the prostate cell culture.

2. A method according to claim 1, wherein the gene is the VDUP-1 gene.

3. A method according to claim 1, wherein the transcription and/or translation product is VDUP-1 or a functional equivalent thereof.

4. A method according to claim 1, wherein the transcription and/or translation product is VDUP-1-mRNA.

5. A method according to claim 1, wherein the gene is a reporter gene.

6. A method according to claim 1, wherein the hyperproliferative disorder is cancer or hyperplasia.

7. A substance for use in the treatment of hyperproliferative disorders of the prostate characterized by being selected by a method according to claim 1.

8. A substance according to claim 7, selected from steroid receptor ligands and compounds that influence the JAK-STAT-SOCS pathway.

9. A substance according to claim 7, characterized in that the hyperproliferative disorder is cancer or hyperplasia.

10. A pharmaceutical composition for use in the treatment of hyperproliferative disorders of the prostate characterized in that it comprises at least one substance according to claim 7 as an active ingredient.

11. A method of therapeutic treatment of hyperproliferative disorder of the prostate in a mammal in need of such treatment, comprising administering to the mammal in need an effective amount of a substance according to claim 7.

12. A gene encoding VDUP-1 or a functional equivalent thereof for use as a medicament.

13. A pharmaceutical composition for use in the treatment of hyperproliferative disorders of the prostate characterized in that it comprises a gene encoding VDUP-1 or a functional equivalent thereof.

14. A method of therapeutic treatment of hyperproliferative disorder of the prostate in a mammal in need of such treatment, comprising administering to the mammal in need an effective amount of a gene encoding VDUP-1.

15. A vector capable of expressing functional VDUP-1 or variants thereof, for use as a medicament.

16. A method of therapeutic treatment of hyperproliferative disorder of the prostate in a mammal in need of such treatment, comprising administering to the mammal in need an effective amount of a vector capable of expressing functional VDUP-1.

17. A method of diagnosis of a hyperproliferative disorder of the prostate in a mammal by assessing the content of the transcription and/or translation products of at least one of the genes encoding ezrin, thioredoxin, peroxiredoxin 5, cyclin-dependent kinase 4, growth response protein CL6, defender against cell death 1, CD24, osteoactivin, vitamin D-upregulated protein 1, placental growth factor gene 1+2, Gata-4, duffy blood antigen, interferon-inducible 17-kDa membrane protein, 14-3-3 protein, CD9 antigen (p24), Ste-20 related kinase, ephrin type A receptor 5 precursor, programmed cell death factor 4 and DNA-methyltransferase 3a, in cells obtained from the prostate of said mammal.

18. A method according to claim 17, wherein the gene is the VDUP-1 gene.

19. A kit for use in a method of diagnosis of a hyperproliferative disorder of the prostate in a mammal, comprising an agent for assessing the level of the transcription and/or translation products of at least one of the genes encoding ezrin, thioredoxin, peroxiredoxin 5, cyclin-dependent kinase 4, growth response protein CL6, defender against cell death 1, CD24, osteoactivin, vitamin D-upregulated protein 1, placental growth factor gene 1+2, Gata-4, duffy blood antigen, interferon-inducible 17-kDa membrane protein, 14-3-3 protein, CD9 antigen (p24), Ste-20 related kinase, ephrin type A receptor 5 precursor, programmed cell death factor 4 and DNA-methyltransferase 3a.

20. A method of therapeutic treatment of hyperproliferative disorder of the prostate in a mammal in need of such treatment by increasing the content of transcription and/or translation product of the VDUP-1 gene of prostate cells in said mammal.

21. A method of therapeutic treatment according to claim 20, wherein the translation product is VDUP-1.

22. A method according to claim 21, wherein the content of VDUP-1 is increased by administration, to said mammal, of at least one compound capable of inhibiting the break-down of VDUP-1 within the prostate cells of said mammal.

23. A composition for treatment of prostate cancer comprising anti-androgens and vitamin D.

24. A composition for prevention of prostate disease comprising low levels of androgen and antioxidants like vitamin E.
